(19) United States
(12) Patent Application Publication
(10) Pub. No.: US 2020/0309872 A1
(43) Pub. Date: Oct. 1, 2020

(54) APPARATUS AND METHOD OF AIR-SUSPENDED BIOFABRICATION OF TISSUE-ENGINEERED ORGAN CONSTRUCTS AND CONGLOMERATES OF SPHEROIDS, CELLS AND OTHER BIOLOGICAL OBJECTS BY USING MAGNETIC FIELD

(71) Applicant: VIVAX BIO, LLC, New York, NY (US)

(72) Inventors: Vladislav Parfenov, Podolsk (RU); Stanislav Petrov, Pushkino (RU); Yusef Khesuani, Moscow (RU); Frederico David Alencar de Sena Pereira, Vitória da Conquista (BR); Elizaveta Kudan, Moscow (RU); Vladimir Mironov, Moscow (RU); Pavel Karalkin, Moscow (RU); Elena Bulanova, Moscow (RU); Alexander Ostrovskiy, Moscow (RU); Yakov Balakhovsky, New York, NY (US); Elizaveta Nezhourina, Moscow (RU); Anna Gryadunova, Ulyanovskaya oblast region (RU)

(21) Appl. No.: 16/832,446

(22) Filed: Mar. 27, 2020

Related U.S. Application Data
(60) Provisional application No. 62/825,381, filed on Mar. 28, 2019.

(51) Int. Cl. G01R 33/12

(52) U.S. Cl. CPC G01R 33/1261; C12M 35/06; G01R 33/1269

(57) ABSTRACT

The present invention generally relates to biofabrication technology and, more particularly, to systems and methods for manufacturing three-dimensional constructs made of various materials using scaffold-free, nozzle-free and label-free magnetic levitation in non-toxic paramagnetic medium. The essence of the method consists of rapid levitational assembly in the construct's heterogenous magnetic field from various materials, such as, for example, tissue spheroids, single-cell suspension, microorganisms, peptides, potassium phosphate granules, which are chaotically distributed in the volume of culture medium. The construct is formed in a specific area where a magnetic trap is formed as a result of the combined forces of gravitational and magnetic fields. In this area, the gravitational pull is compensated and particles of material are forced together. The introduced technology can become a powerful biofabrication tool that enables rapid assembly of various three-dimensional constructs, including biological tissues and organs.

### FIELD OF THE INVENTION

The present invention generally relates to biofabrication technology and, more particularly, to systems and methods for production of constructs from various materials using scaffold-free, nozzle-free and label-free magnetic levitation in non-toxic paramagnetic medium.

### BACKGROUND OF THE INVENTION

At the present time the application of tissue spheroids as building blocks for creating tissue-engineered and organ constructs is growing increasingly popular. This is due to the fact that tissue spheroids possess wide array of advantages in comparison to single cells. In today's existing additive technologies various dispensers are used in layer-by-layer deposition. Moreover, different temporary scaffolds such as hydrogels, polymers, metallic cores, etc. are essential to support spheroids at certain space points. However, applying supportive structures goes hand-in-hand with several difficulties involving increase of printing time. The impossibility to deposit spheroids in direct contact with each other enable the creation of branching tubes inside organ constructs. An alternative approach is a magnetic biofabrication using spheroids containing magnetic nanoparticles, also known as labels. However, taking into the account that potentially toxic concentrations of nanoparticles are used to achieve the sufficient magnetic force, the application of this technique is very limited. Our technology and device provide scaffold-, nozzle- , label-free fast biofabrication of organ constructs by using heterogenous magnetic field specifically created for this purpose. This magnetic field acts like a temporary physical scaffold, termed “scaffield”. In addition to this, the described technology and device can be applied not only for tissue spheroids, but for single-cell suspension as well as for other materials.

### SUMMARY OF THE INVENTION

The present invention provides a method of magnetic fabrication of third-dimensional biologic, organic, non-organic and/or tissue-engineered constructs formed from compatible materials via magnetic levitation in inhomogeneous magnetic field with the region of lowest field intensity in the centre and chaotically distributed in the active volume of paramagnetic culture medium. The method comprises the step of when active volume is placed in the centre of inhomogeneous magnetic field.

In some embodiments, for example, but not limited to tissue spheroids, single-cell suspension or microorganisms such as protozoa, fungi, microalgae, bacteria, and/or their consortiums can be used as biomaterial.

In some embodiments, for example, but not limited to highly molecular organic compounds such as peptides or proteins can be used as organic material.

In some embodiments, for example, but not limited to calcium phosphate granules can be used as non-organic material.

According to the invention the paramagnetic medium contains gadolinium compounds (Gd3+) in various concentrations.

According to the invention in one embodiment, inhomogeneous magnetic field is created via magnetic system that consist of at least two annular permanent magnets oriented towards each other by the same poles.

In addition, there is the straight hole in magnetic system located perpendicularly to the axis of magnetic rings. This hole is used for observation of fabrication process via at least two digital cameras, light source and lens system.

The method may further comprise the step of the magnetic system placement in incubator to ensure compatible temperature conditions. The optimal temperature may vary from 0 to 40° C. depending on the material.

According to the invention in one embodiment, inhomogeneous magnetic field is created via Bitter magnets. It should be noted that magnetic density of Bitter magnets varies from 2 to 32 T.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 shows experimental magnetic installation.

FIG. 2 shows biofabrication of tissue spheroids for 3D microtissue construct assembly.

FIG. 3 illustrates estimation of toxic effect of Gd3+ on chondrospheres morphology at different Gd3+ concentrations.

FIG. 4 illustrates the mechanical properties of tissue spheroids during the testing.

FIG. 5 shows fusion of tissue spheroids as function of time/

[symbol]

FIG. 6 shows levitation assembly of constructs in a magnetic field.

FIG. 7 shows computer simulation and histology analysis that demonstrates formation of gaps and empty spaces between tissue spheroids as result of their incomplete fusion within constructs.

### DETAILED DESCRIPTION OF THE PREFERRED EMBODIMENT

This invention describes a novel scaffold-free, label-free and nozzle-free biofabrication technology. The essence of the method consists of rapid levitational assembly in the construct's heterogenous magnetic field from the material such as: tissue spheroids; cells; cells with non-organic nanoparticles; bacteria; fungi; microalgae; microorganisms; highly molecular organic and non-organic compounds (for example, proteins, polymers, peptides, granules and macro-, micro-, nanoparticles from calcium phosphate, metals, etc.) that are chaotically distributed in the volume of culture medium. The construct is formed in a specific area where a magnetic trap is formed as a result of the combined forces of gravitational and magnetic fields. In this area the gravitational pull is compensated and the material is forced together. The assembled construct can have the form of a sphere, toroid, ellipsoid and many others, all of which are defined by the configuration of the magnetic field. However, the magnetic field configuration is defined by the magnetic system. For example, in case of using the magnetic system scheme from Fig. Xa the construct will be shaped as ellipsoid of rotation, magnetic system scheme from Fig. Xa—the construct will have elongated form. This construct assembly method can be called formative. When the assembly phase is finished, the levitating construct continues to remain suspended and under the influence of the magnetic field until the fusion of the material is complete.

The technology requires generation of heterogenous magnetic field, which allows depositing chaotically distributed material into working area.

In one embodiment, heterogenous magnetic fields are created by applying several constant magnets of defined shape and their relative arrangement. For example, neodymium magnets can be used.

In another embodiment, creater gradient of magnetic field, hence, larger working area can be achieved by using the system of superconductive magnets or Bitter magnets.

The principle of operation implies the creation of a local microgravity zone where the effects of all acting on diamagnetic objects forces are compensated. In preferred embodiment setup, the directed upward Archimedes force, the directed downward force of gravity, and the directed toward the local minimum of the magnetic field magnetic force simultaneously act on the object under static conditions. The magnetic forces moment appears only if the magnetic field is non-homogeneous.

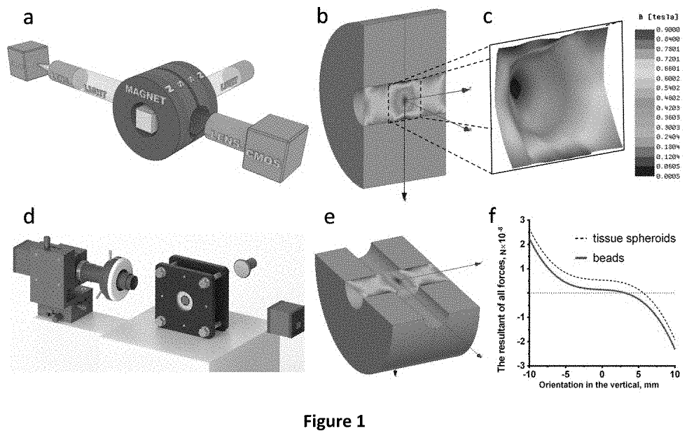

As a result, diamagnetic objects will be pushed out into an area with lower field strength (magnetic trap) under the action of a magnetic force. In Earth's gravity condition the equilibration of objects occurs at a certain distance from the local minimum of the magnetic field, whereas in zero gravity, this process occurs exactly in the area of the local minimum. The magnetic gradient while facing the same direction as gravity force should be above 1,3 T/cm to provide levitation in gravity condition. All of this is accomplished while applying culture media containing paramagnetic salts, such as gadolinium salts. The FIG. 1 shows magnetic system as well as the horizontal and vertical cross-section of the magnetic field.

The magnetic field gradient necessary for levitating, for example, the tissue spheroids is achieved via particular form and arrangement of the magnets, assuming that the paramagnetic concentration in the medium is bellow toxicity level. Namely the magnets are arranged so that surfaces with the same magnetic pole come in contact with one another. As a result of such configuration, the magnetic gradient reaches the value of 2.2 T/cm facing the same direction as the gravity.

In the depicted device the architectural complexity and the size of assembled constructs depend on working capacity and the complexity of magnet system.

In one embodiment according the invention, tissue spheroids have been proposed as building blocks in 3D biofabrication. Although, the following is the example of the three-dimensional constructs biofabrication from tissue spheroids, but it is not the limiting example. The method and apparatus of the present invention can also be used, for example, to create three-dimensional constructs from other biological, organic and/or non-organic materials.

Tissue spheroids represent densely packed aggregates of living cells. At first, they were used to design the models of human diseases in vitro and test lead candidates at preclinical stages of drug development. More recently, tissue spheroids were proposed as building blocks for fabrication of human tissues and organs.

Several beneficial advantages for using tissue spheroids as building blocks exist. First, tissue spheroids have a highest theoretically possible cell density comparable with natural tissue. Second, tissue spheroids have a compact rounded shape, which is ideally suitable for their handling, manipulation, transfer, processing and bioprinting. Third, they have a complex internal structure and multicellular composition. Moreover, tissue spheroids can have one or several lumens and even can be pre-vascularized. Finally, they have intrinsic properties for fusion. Tissue fusion is an ubiquitous phenomenon during embryonic development and it is a fundamental principle of rapidly emerging bioprinting and biofabrication technologies based on the use of tissue spheroids as building blocks. Closely placed and directly touching each other, tissue spheroids begin to fuse thus producing complex 3D tissue constructs.

Several approaches exist for tissue and organ biofabrication using tissue spheroids.

Using one method, cell aggregates were placed robotically or manually into 3D scaffolds printed from various biodegradable biomaterials. Another approach involves the spreading of tissue spheroids on electrospun matrices. Furthermore, magnetic forces have been used for 2D patterning of tissue spheroids, biofabricated from cells labeled with magnetic nanoparticles. All described biofabrication approaches are scaffold-based, nozzle-based or label-based. These methods have certain advantages and inherent limitations. For example, robotic 3D bioprinting allows to fabricate complex 3D tissues and organs.

Further, most of the current approaches involve the use of biomaterials or nanomaterials as scaffolds. Conventional magnetic force-driven 2D patterning of tissue spheroids requires prior cell labelling by magnetic nanoparticles, meanwhile a label-free approach for 3D magnetic levitational assembly has been introduced.

The invention in one embodiment of the invention is illustrated rapid assembly of 3D tissue construct using scaffold-free, nozzle-free and label-free magnetic levitation of tissue spheroids.

The label-free levitational diamagnetic assembly strategy could be used as a powerful tool to fabricate soft small living blocks. This strategy allowed for the first time the alignment of living blocks in a paramagnetic suspending media for remote 3D assembly. During reported magnetic levitation experiments Gd³⁺ has been used to paramagnetic suspending media. The paramagnetic Gd³⁺ formulations such as Omniscan have been already approved by FDA for clinical use as a contrast agent for magnetic resonance imaging investigations in humans. However, the high concentrations of Gd³⁺ could be potentially toxic for tissue spheroids and a certain risk exists for osmotic pressure imbalance due to excessive use of ions in paramagnetic medium. Theoretically, these limitations could be overcome by combination of stronger magnetic fields and smaller density differences between building blocks and medium. All this enable self-assembly approaches based on magnetic forces that are particularly significant since i) magnetic forces enable contactless manipulation in 3D; ii) the spatial distribution of the magnetic forces can be designed to vary using arrays of magnets and electromagnets; and iii) a globally applied magnetic field is capable for addressing a large number of components in parallel, iv) self-assembly approaches enable scaffold-free biofabrication of 3D tissue constructs.

The experiments confirmed that relatively weak magnetic fields allow levitational assembly of tissue spheroids only in the presence of toxic Gd³⁺+ concentrations. However, the employing of high gradient magnetic field enables assembly of tissue spheroids at non-toxic concentration of Gd³⁺ in paramagnetic medium.

This invention describes a novel technology of label-free and nozzle-free magnetic levitation of tissue spheroids in non-toxic paramagnetic medium. The following Examples provide a proof of concept for new strategies in tissue engineering. The following Examples should not be construed so as to limit the invention in any way.

Chondrospheres of standard size, shape and capable to fusion have been biofabricated from primary sheep chondrocytes using non-adhesive technology. Label-free magnetic levitation was performed using a prototype device equipped with permanent magnets in presence of gadolinium (Gd³⁺) in culture media, which enables magnetic levitation.

Mathematical modelling and computer simulations were used for prediction of magnetic field and kinetics of tissue spheroids assembly into 3D tissue constructs. First, polystyrene beads were used to simulate the assembly of tissue spheroids and to determine the optimal settings for magnetic levitation in presence of Gd³⁺. Second, the ability of chondrospheres to assemble rapidly into 3D tissue construct in the permanent magnetic field in the presence of Gd³⁺ was proved.

### DEFINITIONS

Definitions of several terms used in this description are given below. If not defined differently herein, technical and scientific terms in this invention have standard meanings common for technical and scientific literature.

As used herein the term “material” refers to any materials (or its combinations) that can be used for tissue-engineered constructs fabrication such as tissue spheroids, single-cell suspension, microorganisms (protozoa, fungi, microalgae, bacteria or its consortiums), highly molecular organic and compounds, potassium phosphate granules (octacalcium phosphate, α-tricalcium phosphate), etc.

The term “construct” refers to inseparable, solid construct formed via magnetic fabrication; depending on the material the construct can be biological, organic, non-organic and/or tissue-engineered.

As used herein the term “tissue spheroids” (or “spheroids”) refers to tissue spheroids that can be formed from various cells' types. For example, spheroids can consist of fibroblasts, chondrocytes, keratinocytes, primary astrocytes, thyrocytes, MMSC (multipotent mesenchymal stromal cells), tumour cell lines (e.g.: human melanoma cells) but not limited to them. In some embodiments of this method different types of tissue spheroids (so consisting from different cells' types) can be used for simultaneous fabrication.

As used herein the term “medium” (“culture medium”) refers to any medium intended for performing of biofabrication, it is chosen due to the type of biological, organic and/or non-organic material used for constructs fabrication.

For example, the alpha-MEM medium can be used for tissue spheroids from keratinocytes, primary astrocytes and human melanoma cells. The DMEM medium can be used for tissue spheroids from fibroblasts, chondrocytes, MMSC and tumour cell lines. The F-12 medium can be used for tissue spheroids from thyrocytes, Chinese hamster ovary cells cultures and hybridoma cells. The RPMI-1640 medium can be used for tissue spheroids from lymphoid cells. The DMEM/F12 medium can be used for tissue spheroids from pancreatic cells. LB medium can be used to cultivate microorganisms.

“Paramagnetic medium” (“paramagnetic culture medium”) is the medium containing paramagnetic for performing biofabrication. Any compounds having paramagnetic properties (so they get magnetization in the direction of magnetic field vector when placed in external magnetic field) can be used as paramagnets. The first-choice paramagnets are those which have no toxic effect on cultured microorganisms such as gadolinium salts and chelates, copper sulphate, manganese chloride, etc. according to the invention. The minimum concentration of paramagnetic is selected to ensure microorganisms levitation in inhomogeneous magnetic field. This concentration depends on the type of material, magnetic field parameters, medium composition, other biofabrication conditions (temperature, etc.). For example, in case of using gadolinium Gd³⁺ salts as paramagnetic its concentration may vary from 0.1 to 5000 mmol depending on the material. In particular embodiments of the invention the gadolinium concentration can be 0.1-50 mmol for fabrication of constructs from tissue spheroids of various cells' types.

As used herein the term “magnetic trap” refers to the geometrical arrangement of magnetic field created for limitation of movements of any object. According to the invention “magnetic trap” is formed in the central part of inhomogeneous magnetic field and it is characterised by escalation of field intensity when the object is moving from the magnetic trap in any direction. The “magnetic trap” is characterized by the minimum parameters of magnetic field intensity that ensures movement and further fabrication of levitated material inside of the magnetic trap.

In the present description and in the summary of invention the terms “includes” and “including” and other grammatical forms are not intended to be considered as limitation but on the contrary it should be used as non-exclusive (like “containing”). As limiting list, you can consider only such phrases as “consists of”.

### Materials and Methods

Reagents

Gadolinium salts (Gadodiamide) were used as paramagnetic for the experiments. Gadodiamide is a paramagnetic gadolinium-based contrast agent (GBCA), with imaging activity upon magnetic resonance imaging (MRI). When placed in a magnetic field, gadodiamide generates a large local magnetic field, which can enhance the relaxation rate of nearby protons.

Dulbecco's modified Eagle's medium (DMEM, cat.# 12491-015), fetal bovine serum (FBS, cat.# 16000-044), antibiotic-antimycotic (cat.# 15240-062), trypsin/EDTA (cat.# 25200-114), phosphate-buffered saline (PBS, cat.# 18912-014) were obtained from Gibco (USA). L-glutamine (cat.# F032) were obtained from Paneco (RF). Glutaraldehyde (cat.# G5882) was obtained from Sigma-Aldrich (USA). CellTiter-Glo 3D kit (cat.# G9682) was purchased from Promega (USA). Omniscan (gadodiamide) was purchased from GE Healthcare (Ireland).

Cell Culture

The primary culture of articular sheep chondrocytes was kindly provided by Dr. N. P. Omelianenko (N.N. Priorov's Central Research Institute of Traumatology and Orthopaedics, Moscow, Russia). Cells were grown in DMEM medium containing 10% FBS, supplemented with antibiotic/antimycotic and 2 mM L-glutamine. The cells were incubated at 37° C. in a humidified atmosphere with 5% CO2 and routinely split at 85-95% confluence. Cell transfer and preparation of single-cell suspensions were performed using mild enzymatic dissociation with a 0.25% trypsin/0.53 mM EDTA solution. Cells were confirmed free of mycoplasma contamination according DAPI (Invitrogen, cat. # D1306) staining protocol.

Formation of Tissue Spheroids using Corning Spheroid Microplates

Tissue spheroids were formed using ultra-low adhesion Corning spheroid microplates (Corning, cat.# 4520) according to the manufacturer protocol. Briefly, monolayer cells with 95% confluence were rinsed by EDTA solution, harvested from the culture flasks by 0.25% trypsin/0.53 mM EDTA and then suspended in cell culture medium. The concentration of the cells was 8×104 per millilitre. 100 μl of cell suspensions were dispensed to the wells of Corning spheroid microplates. Corning spheroid microplates were incubated at 37° C. in a humidified atmosphere with 5% CO2 for 3 days.

Determination of Spheroid Diameter and Roundness Distribution

Tissue spheroids were biofabricated and captured at 3 d day in culture using bright-field imaging at inverted microscope Nikon Eclipse Ti-S, Japan. Spheroid diameters and roundness were measured using Image J 1.48v software (NIH, Bethesda, Md., USA). Briefly, all original grayscale images were converted to simplified threshold images under the same converting condition and the edges of the spheroids were automatically detected. MinFeret's diameters of the detected spheroid edges were measured initially as pixels, and converted to micrometers by comparing to a reference length. Roundness was measured using Image J 1.48v shape descriptor and calculated as 4*area/(π*major axis²).

Estimation of Tissue Spheroids Viability at Different Concentrations of Gadolinium

The viability of tissue spheroids was assessed using the CellTiter-Glo 3D kit according to the manufacturer protocol. Briefly, 3-day-old tissue spheroids with a starting cell number of 8000 cells were placed in 0, 50 and 250 mM Gd3+ concentrations for 24 hours. Then CellTiter-Glo 3D kit was added and luminescence was recorded after 30 min incubation using VICTOR X3 Multilabel Plate Reader (Perkin Elmer, USA).

Mechanical Testing

The mechanical properties of tissue spheroids were measured by a micro-scale parallel-plate compression testing system Microsquisher (CellScale, Canada) with associated SquisherJoy software. Tissue spheroids with a starting cell number of 8000 cells were formed using Corning spheroid microplates. They were cultured 3 days and then placed in 0, 50 and 18 250 mM Gd3+ concentrations for 24 hours prior to mechanical characterization. For mechanical testing spheroids were placed in a PBS-filled bath at 37° C. and compressed to 50% deformation in 20 sec. The microbeams with diameters 152.4 μm (recommended max force 57 mN) and 304.8 μm (recommended max force 917 mN) were employed depending on the stiffness and sensitivity required to measure tissue spheroids from different cell types. The force-displacement data obtained from the compression test were converted to stress-strain curves and the lower portion of the curve (0-20% strain) was used to obtain a linear regression line and estimate the Young's modulus. In each group eight samples of spheroids were measured.

Spheroid Fusion Assay

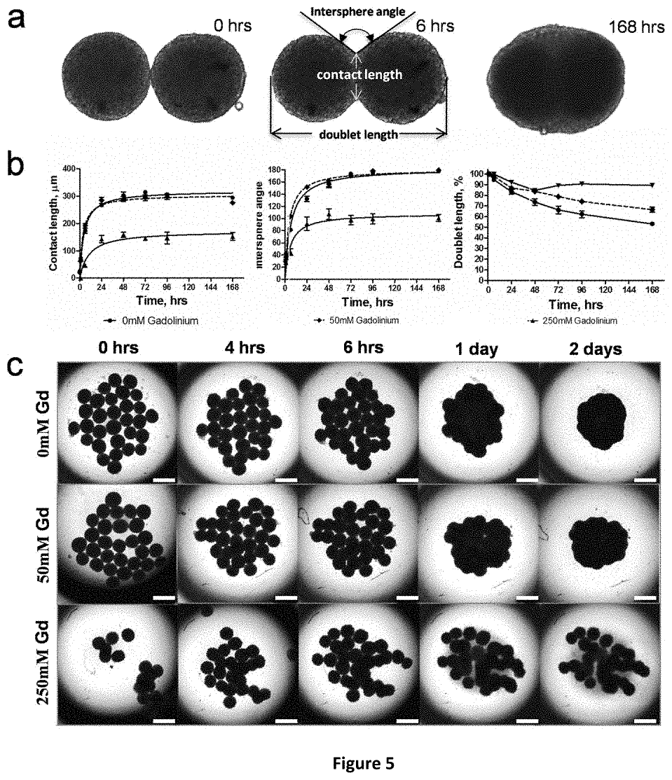

Spheroid fusion assay was performed using Corning spheroid microplates (Corning, cat.# 4520). Pairs of 3-day-old tissue spheroids with a starting cell number of 8000 cells were placed in contact in 0, 50 and 250 mM Gd3+ concentrations and incubated for 7 days. Bright-field images of spheroid doublets were obtained at points 0 h, 4 h, 6 h, 1 day, 2 days, 3 days, 4 days and 7 days using Nikon Eclipse Ti-S microscope. Contact length, intersphere angle and doublet length were measured using Image J 1.48v software (NIH, Bethesda, Md., USA) and plotted as a function of time using GraphPad Prism software (GraphPad Software, Inc., La Jolla, Calif.). Doublet length (FIG. 5) was normalized to initial doublet length. Measurements for each parameter were reported as mean ±S.E.M.

Electrospinning of Polyurethane

Polyurethane was kindly provided by Dr. Xuejun Wen (EG-85A, Lubrizol, USA). Electrospinning of microfibrous polyurethane matrix was performed using commercial Professional Electrospinning Lab Device (Yflow, Spain). Polyurethane was dissolved to 17% concentration in solvents containing 40% N,N-dimethylformamide and 60% tetrahydrofuran.

Scanning Electron Microscopy (SEM)

Tissue spheroids and constructs made from chondrospheres were placed on the surface of electrospun polyurethane matrix and allowed to attach during 6 hours. Subsequently samples were fixed with 2.5% glutaraldehyde/PBS, dehydrated through ethanol series and then were dried in a critical point dryer (HCP-2, Hitachi Koki Co. Ltd., Japan). The samples were transferred on a stub of metal with adhesive surface, coated with gold using ion coater (IB-3, EIKO, Japan) and then observed using the microscope JSM -6510 LV (JEOL, Japan).

Tissue Spheroids Morphology

Tissue spheroids were fixed with 2.5% glutaraldehyde/PBS and then treated with 1% osmium tetraoxide/PBS. The samples were dehydrated by soaking in a series of solutions of increasing concentrations of ethanol, stained with 1% uranyl acetate/70% ethanol and finally embedded to araldite-epon mixture. Semi-thin sections were prepared on a LKB-III ultratome (Sweden), stained with 1% toluidine blue and then examined using a light microscope (Leica DM 2500, Germany) equipped with a digital camera (Leica DFC 290, Germany).

Tissue Construct Histology

The constructs, assembled from chondrospheres in magnetic field, were fixed in 10% buffered formalin (pH 7.4) for 24 hours and then embedded in paraffin with a melting point of 28 +54° C. (Biovitrum, RF). Serial sections with a thickness of 5 μm were made with microtome Microm HMS 740 (ThermoFisher Scientific, USA), routinely stained with hematoxylin and eosin (Biovitrum, RF) and then covered with Bio-Mount medium (Bio Optica Milano S.P.A., Italy) before histological examination.

Estimation of Spheroid Cell Density

Spheroid cell density was estimated by analysis of semi-thin sections of tissue spheroids using Image J 1.48v software (NIH, Bethesda, Md., USA).

Determination of Polystyrene Bead Density

The density of polystyrene beads (Polyscience, Inc. USA, cat.# 64235) was determine by means of equating the force of gravity with buoyancy force. We put polystyrene beads with diameter 170 μm in the container with deionized water, and then the dextrose powder was added to the container followed by the thorough mixing. Gradual adjusting of the dextrose concentration in the solution provided floating of the beads in the water volume without sinking or emerging at the surface. Thus, we obtained the polystyrene beads density equal to 1.0405 g/cm ³.

Surface Evolver Simulation

The fusion behaviour of tissue spheroids was illustrated using the open source software Surface Evolver. Tissue spheroids were approximated and modelled as ball-like liquid droplets of standardized size and volume. The initial positions of closely contacting tissue spheres were random, then evolved by gradient descent to minimize distance between centers subject to centers being at least a diameter apart. The progressive process of fusion of tissue spheres was modeled by iterating gradient descent to minimize the surface energy subject to the constraint of constant spheroid volumes, until movement stopped. The evolution script created interfaces between spheroids where spheroids touch. The configuration reached is a local minimum of energy, but not necessarily a global minimum. The progressive changes in the shape of single spheroids contacted with each other inside forming compacted tissue constructs have been visualized. The simulations have been performed for tissue spheroids.

Magnetic Experimental Setup

To perform the experiment custom laboratory installation was designed with construction shown on FIG. 1(a, d). FIG. 1a shows schematic diagram of a magnetic installation. The installation consists of 2 CMOS digital cameras (DMK41AU02, The Imaging Source Europe GmbH, Germany), light sources, lens system (Optem ZOOM 70XL, Qioptiq, Germany), which are mounted on 3-axis positioning system assembled from 3 linear stages (TSX-1D, Newport Corporation, USA), magnet holding system and 2 ring-shape NdFeB magnets with cutouts for video capture. The external diameter of magnets is 85 mm; the internal diameter is 20 mm; thickness (height) is 24 mm. Magnets are assembled in such a way that they oriented to each other with the same poles. FIG. 1d shows 3D model of the magnetic experimental setup. This experimental setup was placed in the incubator to ensure the appropriate temperature regime (37° C.) during the process of the constructs assembly.

Non-homogeneous magnetic field with orifice in the center of the magnets was created in the axial hole of the magnetic installation (working zone). The distribution of the magnetic induction values in the working area vertical section (FIG. 1b) is shown in the 3D model graph (FIG. 1c). A transparent glass cuvette with dimensions of 12×12×50 mm we inserted into the hole of the magnet system. The cuvettes were filled with paramagnetic liquid contained diamagnetic particles: polystyrene beads or tissue spheroids. The paramagnetic liquid consisted of the DMEM medium and range of Gd3+ concentration: 0, 50, and 250 mM. The process of levitational assembly for polystyrene beads and tissue spheroids was recorded by two video cameras with 1× and 3× optical magnification. After tissue spheroids assembling, resulted constructs continued to levitate in a magnetic field for 24 hours to full complete the fusion process.

The principle of operation of experimental installation implicates the creation of a local microgravity zone where the effects of all acting on diamagnetic objects forces are compensated. In this setup, the directed upward Archimedes force, the directed downward force of gravity, and the directed toward the local minimum of the magnetic field magnetic force simultaneously act on the object under static conditions. The magnetic forces moment appears only if the magnetic field is non-homogeneous. Then the effective magnetic force, acting on the object in non-homogeneous magnetic field will be equal to the volume integral:

$$F = \frac{{x}^{m}}{2} ∇ ( {B}^{2} ) ,$$

where for paramagnets χ>0, while for diamagnets χ<0. The sign determines the direction of the magnetic force action.

As a result, diamagnetic objects will be pushed out into an area with lower field strength (magnetic trap) under the action of a magnetic force. In Earth's gravity condition the equilibration of objects occurs at a certain distance from the local minimum of the magnetic field.

Computer Simulation of Magnetic Field

The simulation of three-dimensional non-homogeneous static magnetic field in a paramagnetic medium from two permanent magnets we performed using “ANSYS 30 Electromagnetics Suite” software for Maxwell3D. The characteristics of magnetic field used in the simulation were as follows: relative permeability paramagnetic medium 1.00004135; and material grade of NdFeB magnet N38 (VG=1.21 TL).

Molecular Dynamics (MD) Simulation of Dynamics of the Polystyrene Particles in the Cusp Magnetic Trap

We assumed that all 3 particles in MD calculations are spherical, having the same size and mass mₚ. For modeling dynamics of the diamagnetic polystyrene particles in the cusp magnetic trap, we have numerically solved the system of the Newton equations for all the particles (1<k<N)

$${m}_{p} \frac{{d}^{2} {r}_{k}}{d {t}^{2}} = {\sum }_{l} F ( \vert {r}_{k l} \vert ) \frac{{r}_{k l}}{\vert {r}_{k l} \vert} + {F}_{k B} + {f}_{k} + {f}_{g} + {f}_{b}$$

where rₖ is the position of the center of a particle k, rₖₗ=rₖ−rₗ The first term in the right-hand part is the Lennard-Jones interaction with other particles, the second term is the interaction with the magnetic field of the trap (FIG. 1c), the third term is the force of viscous friction against the continuum liquid which is determined by the Stokes formula fₖ=−3πd ηuₖ, η is the viscosity of Gd3+ suspension, d is the particle diameter and uₖ=drₖ/dt is the particle velocity relative to the stationary liquid, the fourth and the fifth terms is a force of gravity f_{g} 13 and buoyancy force f_{b} acting on the particle. Numerical simulation was performed for N=100.

The simulation was performed for polystyrene beads and tissue spheroids with densities ρ=1.0405 g/cm³ and ρ=1.05 g/cm³, respectively. At the first step, particles were distributed randomly within the computational domain with initial zero velocity. The computational domain corresponds to the internal volume of the experimental cell—12×12×47 mm.

Data Analysis

Statistical data was analyzed using GraphPad Prism software (GraphPad Software, Inc., La Jolla, Calif.) and represented as mean ±S.E.M. The Analysis of Variance (ANOVA) test was used to find the significant differences between the means of the three groups with P<0.0001.

### Experimental Results

Magnetic Experimental Setup

A magnetic installation (FIG. 1d), which generates a magnetic field in the active volume with a very high (calculated value is up to 2.2 T/cm) gradient along the vertical axis, was created. The distribution of magnetic induction, created by the system of magnets, is shown in FIG. 1 (b, c, e). FIG. 1b shows vertical cross section of the magnetic field. FIG. 1c shows vertical distribution of the vector of the magnetic induction at the working area. FIG. 1e shows horizontal cross-section of the magnetic field at the working area. Method molecular dynamics modeling was used to estimate the time, shape and height of constructs assembly in a magnetic field. The fusion dymanics modeling resulted in the graph (FIG. 1f), which shows the relation between the projection on the vertical axis of the resultant force, acting on particles (tissue spheroids and polystyrene beads) during the experiment, and the vertical coordinate in the laboratory installation system of axes. The resultant force acting on the vertical axis y on the diamagnetic bodies, with the resultant force equal to zero, conditions of levitation are created (3.9 mm for polystyrene beads, 5.1 mm for tissue spheroids).

Polystyrene beads were used to test the operations and debugs for all modes. They proved to be sufficiently good physical analogues of tissue spheroids for such parameters as magnetic susceptibility and density. The use of tissue spheroids for routine testing of assembly modes was time and cost consuming, so we used polystyrene beads as their substitutes. Indeed, beads and tissue spheroids have the similar parameters of density and magnetic susceptibility. After initial determination of the optimal magnetic field configuration, we continued our experiments with tissue spheroids.

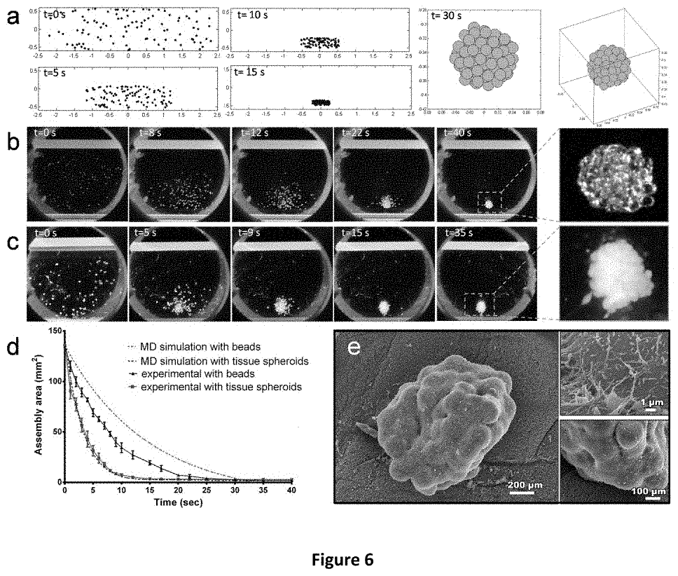

The construct's center of mass corresponds to the point, where the resultant force turns into zero, thus forming levitation point. According to the graph (FIG. 1f) this point has the following coordinates in the vertical axis: 3.2 mm for polystyrene beads and 5.6 mm for tissue spheroids. The vertical coordinate of construct's center of mass was calculated by means of video analysis. The coordinates, defined in such way were as follows: 3.9 mm for polystyrene beads, 5.1 mm for 15 tissue spheroids (FIG. 6).

FIG. 6 shows: (a) MD simulation of the chondrospheres dynamics in the cusp magnetic trap. The step by step assembly of construct from polystyrene beads (b) and chondrospheres (c). (d) The kinetics of simulated and experimental construct assembly from polystyrene beads and chondrospheres. (e) Scanning electron microscopy image of chondrospheres construct assembled in a magnetic field during 24 hours. Inserts demonstrate typical chondrocyte surface structures including multiple microvilli at high magnification. Results of simulation are in a good agreement with experiments.

Biofabrication of Tissue Spheroids

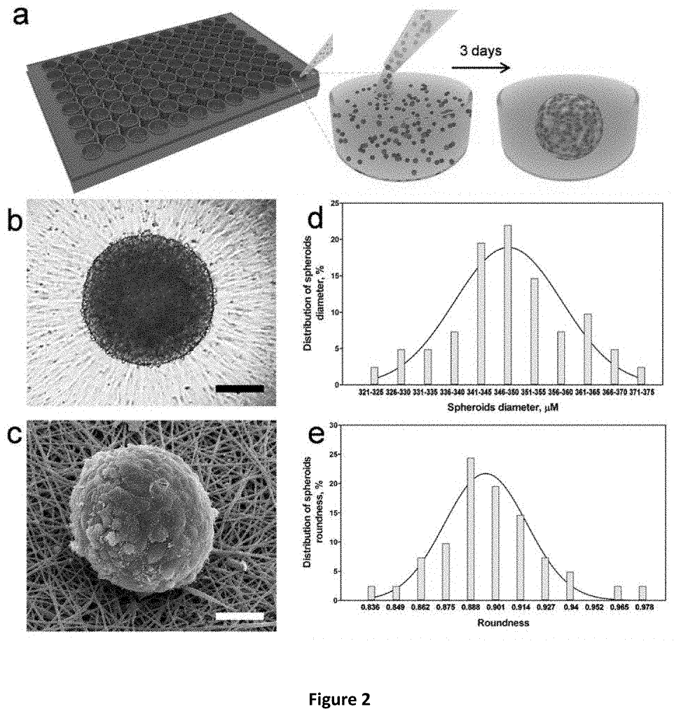

Tissue spheroids were formed from primary sheep chondrocytes using ultra-low adhesion Corning spheroid microplates (FIG. 2a). Their morphology was estimated using bright-field microscopy and SEM. 3-day-old spheroids were used for construct biofabrication.

Tissue spheroids had well-defined edges and spherical shape at 3 d day in culture (FIG. 2b). FIG. 2b shows phase-contrast image of chondrosphere biofabricated from primary sheep chondrocytes. Scale bar—100 μm.

In order to investigate morphology and surface characteristics of tissue spheroids they were analyzed by SEM (scanning electron microscopy). FIG. 2c shows scanning electron microscopy image of chondrosphere on the surface of electrospun polyurethane matrix. Scale bar—100 μm.

The layers of closely packed cells formed the surface of spheroids. Standard size and shape of tissue spheroids is an essential prerequisite for their use in bioprinting as building blocks. Spheroid diameters (FIG. 2d) and roundness (FIG. 2e) were measured after 3 days of culture. FIG. 2d and FIG. 2e show the distribution of chondrospheres diameter, n=192, and the distribution of chondrospheres roundness, n=192.

The average spheroid diameter was 346±16.58 μm. The average spheroid roundness was 0.907±0.047. The standard deviations were <10% of the mean value for both diameter and roundness. These results revealed that tissue spheroids had uniform size and shape and could be used for biofabrication of three-dimensional tissues.

Histological Analysis and Viability of Tissue Spheroid

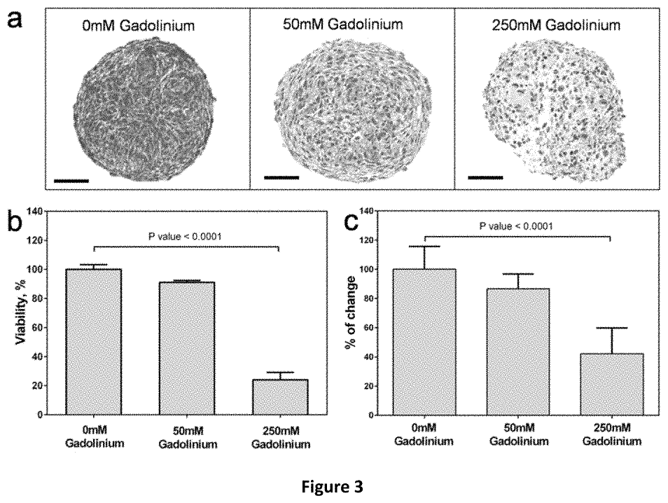

A histological analysis on semi-thin sections of 3-day-old tissue spheroids was performed in order to estimate possible toxic effects and to determine an optimal nontoxic Gd3+ concentration (FIG. 3a). FIG. 3a shows the inner structure of chondrospheres at different Gd3+ concentrations. Toluidine blue staining revealed severe toxic effect on chondrospheres morphology at 250 mM Gd3+. Scale bar—100μm. In non-treated conditions, chondrospheres demonstrated high cell density, swirl-like cell arrangement and intensive staining of cytoplasm. In 50 mM Gd3+ solution cell density did not change but cytoplasm staining was slightly reduced. Finally, at toxic 250 mM Gd3+ concentration, cell density dramatically reduced, that led to manifestations of cell death and apoptosis in form of pycnotic nuclei and accumulation of extracellular debris.

The viability of cells within tissue spheroids was analyzed by measuring the luminescent signal generated by luciferin-luciferase interconnection as a function of cytoplasmic ATP concentration. FIG. 3b shows that tissue spheroids in 50 mM Gd3+ solution remained viable after 24 hours of incubation, while 250 mM Gd3+ concentration showed significant toxic effect. The highly statistically significant difference (p<0.0005) in cell density corresponded to 85% in 50 mM Gd3+ solution compared to 100% in 0 mM Gd3+ solution, while the cell density at 250mM Gd3+ concentration was 40% (FIG. 3c). Thus, cell counting showed that the increase of Gd3+ concentration leads to the reduction in the cell density.

The Mechanical Properties of Tissue Spheroids

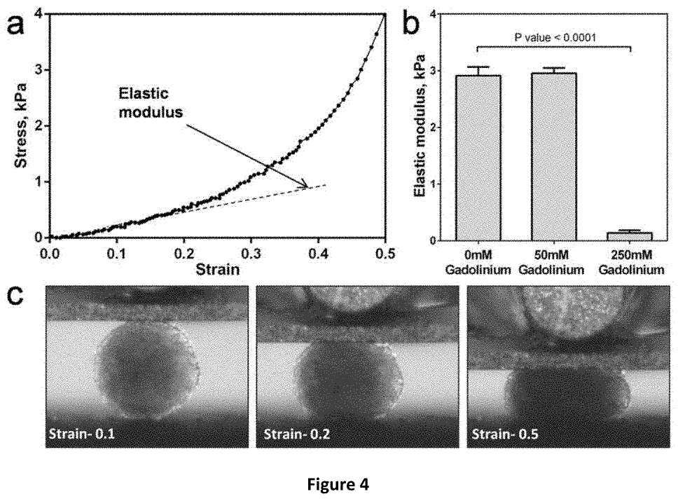

The influence of gadolinium Gd3+ on mechanical properties of tissue spheroids was estimated by tensiometry using parallel plates modification. As shown in FIG. 4, the mechanical properties of spheroids strongly depended on concentration of gadolinium Gd3+. The values of elastic modulus of spheroids in 0 and 50 mM Gd3+ solutions were 2.91±0.15 kPa and 18 2.95±0.1 kPa, correspondingly. The increase of Gd3+ concentration to 250 mM resulted in decrease of elastic modulus value to 0.14±0.05 kPa. This observation can be explained by low viability of tissue spheroids in 250 mM Gd3+ solution.

FIG. 4a shows the stress-strain diagram of chondrosphere obtained from the compression load-displacement curve. The sharp rise of the curve in the area of high strain values is associated with an increase of the tissue spheroid cross section. FIG. 4c shows the stages of chondrosphere compression process between two parallel plates. When compressing up to 20% of the original diameter, an increase of the cross section does not occur. The stress-strain curve changes practically linearly before this strain; in this section we calculated the Young's modulus. According to results, the nontoxic concentration 50 mM Gd3+ in the growth medium does not affect the tissue spheroids Young's modulus (elastic modulus for chondrospheres) at all, which apparently means no significant changes in the internal structure of the tissue spheroid (FIG. 4b), n=12. Finally, at a toxic concentration 250 mM Gd3+, the Young's modulus sharply decreases by ˜93%, indicating that at a given concentration of gadolinium salts irreversible changes occurred within the cells that clearly affects the mechanical properties of the tissue spheroid.

The Fusion of Tissue Spheroids

To investigate the fusion of tissue spheroids at different Gd3+ concentrations, the pairs of spheroids were placed in contact with each other in the same well of low adhesion microplate and allowed to fuse for 7 days. The kinetics of tissue spheroid fusion was monitored and analyzed daily at bright-field under inverted microscope. Contact length, intersphere angle and doublet length were measured. Morphological parameters measured to characterize fusion stage were shown on FIG. 5a, which shows phase-contrast images after 0 h, 6 h and 24 h of fusion for two chondrospheres. FIG. 5b shows Gd3+ effect on the kinetics of contact length, intersphere angle and doublet length change. As shown in FIG. 5b, for spheroid pairs in 0 and 50 mM Gd3+ solutions contact lengths and intersphere angles increased as a function of time and doublet lengths shortened gradually from first day in culture. Spheroid doublet in 0 mM Gd3+ solution shortened faster than spheroid doublet in 50 mM Gd3+ solution. At the seventh day of incubation the intersphere angle increased up to 179° indicating complete spheroid fusion. Spheroid pair in 250 mM Gd3+ solution continued to fuse during first 24 hours and then no further progressive aggregation was observed. This observation confirmed also the high cytotoxicity of 250 mM Gd3+ concentration.

To check the possibility of microtissue construct biofabrication at different concentrations of Gd3+, 30 chondrospheres were placed in the same well of non-adhesive microplate and allowed fusion for 48 hours (FIG. 5c). FIG. 5c shows phase-contrast images with Gd3+ effect on the fusion of 30 tissue chondrospheres placed in ultra-low adhesive microplate. Scale bar −500 μm. At 0 mM and 50 mM Gd3+ concentrations the formation of compact constructs was observed after 2 days of incubation, while no constructs were formed in 250 mM Gd3+ solution.

Assembly of Constructs in a Magnetic Field

Construct assembly was performed in magnetic field using two types of building blocks. The time length for tissue spheroids assembly was shorter by 20% ±1.5% compare to one for polystyrene beads assembly due to the difference in the magnetic susceptibility of diamagnetic materials. Meanwhile, the height of the assembled constructs was lower by 1.2 mm±0.2 mm because the spheroids density is higher than the polystyrene beads density, and, as a result, the gravity force acts more strongly on them. It means that the force compensation will occur in the area where the magnetic force is higher, that is closer to the magnet surface. In another word, the gradient of magnetic field in the working zone does not vary linearly as it is higher near the surface of the magnet. The density of the chamber medium was assumed ρ=1.015 g/cm. Taking into account the near-spherical configuration of the second magnetic field configuration in the trap it can be assumed that the final form of the cluster obtained in MD simulation should be an oblate body with relation between the semiaxes a_{y}/aᵣ=1.

In MD simulations for the formation of cluster from polystyrene particles with diameter d=170 μm and tissue spheroids of diameter d=350 μm, it was set χ=−0.65*10⁻⁶ cm³/g and χ=−0.72*10⁻⁶ cm³/g, respectively. For sufficiently large N the shape of the cluster became almost independent of N, and the ratio of the thickness and diameter of the cluster H_{c}/D_{c} was close to the theoretical value of the relation between the semiaxes a_{y}/aᵣ=1. Experimental values of the ratio H_{c}/D_{c} for polystyrene and tissue spheroids were almost equal and corresponded to the theoretical values.

Configurations of the polystyrene beads and tissue spheroids clusters obtained experimentally and in MD simulation are shown in FIG. 6a. The shape and size of the clusters were similar. Inside the formation area, the force of gravity acted on the particles was counterbalanced by the magnetic force of the trap and the buoyancy force. The center of the mass for the polystyrene particles structure formed by MD simulation located by a distance 3.5 mm downward from the center of the trap. For tissue spheroids, this displacement was up to 4.7 mm.

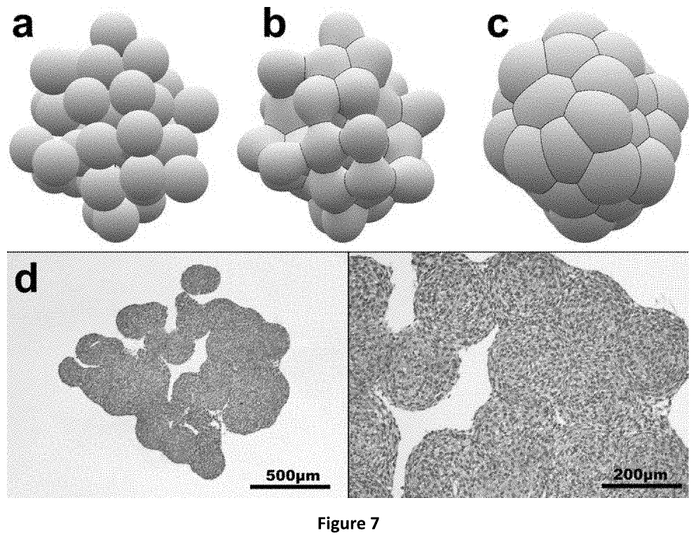

Scanning electron microscopy analysis proved the fusion of tissue spheroids into the compact 3D tissue construct (FIG. 6e). Despite the presence of some grooves and pits corresponding to separate chondrospheres, the ongoing tissue fusion process was obvious because of gradual smoothing of the construct surface Mathematical modeling and computer simulation using Surface Evolver software demonstrated that the formation of grooves is 16 unavoidable in case of random packing (FIG. 7a,b,c). Thus, the formation of holes and empty space could be explained by either the incomplete fusion process or by a chondrospheres rigidity. Experiments involved softer tissue spheroids such as embryonic mouse thyroid gland explants showed complete fusion of tissue spheroids in rounded and compact tissue construct without any holes and empty space.

Computer simulation and histology analysis demonstrated formation of gaps and empty spaces between tissue spheroids as result of their incomplete fusion within constructs were shown on FIG. 7. FIG. 7: (a) Initial touching of tissue spheroids with each other. (b) Intermediate stage of tissue spheroids fusion. (c) The theoretical resulting construct due to tissue spheroids fusion.

Histological analysis was performed to visualize chondrospheres status and fusion in biofabricated tissue construct (FIG. 7d). It was demonstrated that viable chondrocytes were tightly packed inside tissue spheroids. The spheroids fusion kinetics within biofabricated 3D tissue construct was different. As results, there were some areas of complete fusion and there were some gaps or incomplete fusion in tissue which probably reflects random initial packing of chondrospheres. Similar void areas have been shown previously during cell aggregates fusion on pre-stretched electrospun synthetic matrices and also during fusion of chondrospheres biofabricated from cells labeled with iron oxide magnetic nanoparticles.

Conclusion

So experimental results demonstrate the successful rapid biofabrication of millimeter scale microtissue constructs from chondrospheres by magnetic levitational assembly, as one of the variants of invention embodiment. The method from this invention (taking into account the principles from this description with the necessary modifications) can also be used for the magnetic fabrication of three-dimensional tissue-engineered constructs from other materials such as tissue spheroids of various cell types, single-cell suspension, microorganisms, highly molecular organic compounds, potassium phosphate granules, etc.

According to preferred embodiment of this invention, an optimal form of permanent magnets was designed and mutual emplacement using the MD simulation that allowed to perform levitational assembly with nontoxic concentration of the paramagnet in the culture medium were estimated. The rate of construct assembly and its geometrical shape were pre-calculated. As a result, a rather simple but original magnetic system was created, which provided generation of a magnetic field with a gradient of up to 2.2 T/cm with a local minimum at the center of the working area. In the course of an experiment, the data on the construct assembly kinetics and its spatial characteristics, which confirmed the validity of the calculated magnetic field distribution in the magnetic system working area, were obtained.

The data demonstrated that the use of permanent magnets with defined shape and relative position significantly accelerates assembly of both polystyrene beads and tissue spheroids. Quantitative estimation of cytoplasmic ATP production, histological analysis and confirmed tissue spheroids capacity for fusion have confirmed the viability of chondrospheres exposed to 50 mM of Gd3+ in course of magnetic levitation.

Scaffold-free, label-free and nozzle-free biofabrication technology used magnetic levitational assembly has been demonstrated. 3D living tissue constructs have been biofabricated from chondrospheres in paramagnetic medium using non-toxic Gd3+ concentration. The introduced technology can become a powerful biofabrication tool that enables rapid assembly of various tissues and organs.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. The method of magnetic fabrication of three-dimensional biologic, organic, non-organic and/or tissue-engineered constructs via magnetic levitation in inhomogeneous magnetic field with the region of lowest field intensity in the centre and chaotically distributed in the active volume of paramagnetic culture medium, thereby, active volume should be placed in the centre of inhomogeneous magnetic field.

2. The method of claim 1 wherein the biomaterial is represented by tissue spheroids, single-cells, cells including non-organic nanoparticles, microorganisms such as protozoa, fungi, microalgae, bacteria, and/or their consortiums; organic material is represented by highly molecular organic compounds such as peptides or proteins, polymers; non-organic material is represented by granules or macro-, micro-, nanoparticles from calcium phosphate, polymers, paramagnetic and diamagnetic metals.

3. The method of claim 1 wherein the paramagnetic properties of the medium are provided by the presence of gadolinium.

4. The method of claim 1 wherein the inhomogeneous magnetic field is created using a magnetic system consisting of at least two annular permanent magnets oriented towards each other by the same poles.

5. The method of claim 1 wherein the magnetic levitation fabrication is performed in incubator with specified temperature conditions.

6. The method of claim 5 wherein the specified temperature conditions are chosen depending on material for the construct.

7. The method of claim 1 wherein the inhomogeneous magnetic field is created using Bitter magnets.

8. The method of claim 7 wherein the magnetic density of Bitter magnets varies from 2 to 32 T.

9. The device for magnetic fabrication of three-dimensional biologic organic, non-organic and/or tissue-engineered constructs consists of magnetic system of at least two annular permanent magnets oriented towards each other by the same poles with contacting surfaces. It is built with the possibility to place the active volume of the paramagnetic medium with chaotically distributed biologic, organic, non-organic materials. The magnets' sizes are chosen due to necessity of inhomogeneous magnetic field with the gradient not exceeding 1.3 T/cm. Moreover, there is the straight hole in magnetic system located perpendicularly to the axis of magnetic rings. This hole is used for observation of fabrication process via at least two digital cameras, light source and lens system.

10. The method of claim 9 wherein the magnetic system is placed in incubator to ensure compatible temperature conditions.

11. The method of claim 9 wherein the magnetic system is represented by Bitter magnets.

12. The method of claim 9 wherein the magnetic system is represented by superconducting magnets.

13. The method of claim 9 wherein the magnetic system is represented by the combination of Bitter magnet and superconducting magnet.

14. The method of claim 11 wherein the magnetic density of Bitter magnets varies from 2 T to 40 T.

15. The method of claim 12 wherein the magnetic density of superconducting magnets varies from 2 T to 20 T.

16. The method of claim 13 wherein the magnetic density of combined magnetic system varies from 10 T to 60 T.
